(19) United States
(12) Patent Application Publication
(10) Pub. No.: US 2024/0315571 A1
(43) Pub. Date: Sep. 26, 2024

(54) A BIO-PARAMETER MEASURING SYSTEM

(71) Applicant: ESSENCE SMARTCARE LTD., Herzlia Pituach (IL)

(72) Inventors: Haim Amir, Ramat Hasharon (IL); Barak Katz, Petach Tikva (IL); Ohad Amir, Herzlia Pituach (IL)

(21) Appl. No.: 18/269,912

(22) PCT Filed: Dec. 23, 2021
(86) PCT No.: PCT/IL2021/051531

§ 371 Date: Jun. 27, 2023

(30) Foreign Application Priority Data
Dec. 29, 2020 (GB) 2020688.4

(51) Int. Cl. A61B 5/0205; A61B 5/00; A61B 5/0507; A61B 5/1455

(52) U.S. Cl. CPC A61B 5/02055; A61B 5/6898; A61B 5/7221; A61B 5/742; A61B 5/0507; A61B 5/14552

(57) ABSTRACT

The disclosure relates to a bio-parameter measuring system (1) comprising: a first bio-parameter measurement device (2) for performing a first bio-parameter measurement of a person without physical contact with the person (5); an assessment module (3) configured to: receive first measurement data from the first bio-parameter measurement device (2); and, determine whether a second bio-parameter measurement is required on the basis of the first measurement data; a communication module (4) configured to: upon a positive determination that a second bio-parameter measurement is required, notify a user (6) that a second bio-parameter measurement is required; and, receive a validation that the user (6) has undertaken the second bio-parameter measurement.

### RELATED APPLICATION/S

This application claims the benefit of priority of Great Britain Patent Application No. 2020688.4 filed on 29 Dec. 2020, the contents of which are incorporated herein by reference in their entirety.

### TECHNICAL FIELD

This invention relates to a bio-parameter measuring system and method for determining one or more bio-parameters of a person.

### BACKGROUND

Being able to identify the signs of ill health in a person at an early stage can be beneficial for treatment of many diseases and also for limiting the spread of contagious diseases. The measurement of vital signs with various detectors is well known in the art. Many of the detectors used in the art require some form of interaction on the part of the person being assessed. The inconvenience of undertaking bio-parameter measurements may discourage a person from performing such measurements when they should. Conversely, an overly conservative approach may subject a person to an unnecessarily high amount of testing.

The present invention seeks to provide an improved or alternative bio-parameter measuring system and method.

### SUMMARY OF THE INVENTION

The present invention provide a bio-parameter measurement system, a method of measuring a bio-parameter of a person and a computing device according to the appended claims.

The present disclosure provides, in a first aspect, a bio-parameter measuring system comprising: a first bio-parameter measurement device for performing a first bio-parameter measurement of a person without physical contact with the person; an assessment module configured to: receive first measurement data from the first bio-parameter measurement device; and, determine whether a second bio-parameter measurement is required on the basis of the first measurement data; a communication module configured to: upon a positive determination that a second bio-parameter measurement is required, notify a user that a second bio-parameter measurement is required; and, receive a validation that the user has undertaken the second bio-parameter measurement.

Providing a bio-parameter measuring system which can provide a bio-parameter measurement for a person allows a bio-parameter measurement to be obtained in an unobtrusive way. Also, making a determination that a further bio-parameter measurement is needed based on the first bio-parameter measurement allows a further measurement to be made to validate the first bio-parameter measurement or to obtain further information regarding the persons condition. Providing a staged approach in this way, with the first stage being less intrusive and the second stage being potentially more intrusive or interactive but only carried out when necessary provides for an improved system.

A further advantage of the invention is provided by notifying a user of the need for a second bio-parameter measurement and receiving a validation that the requirement for a further measurement has been received and optionally actioned. In this way, the health monitoring of a person can be achieved more readily.

The validation may comprise a confirmation received from the user. The confirmation may be one or more from the group comprising: an acknowledgement that the notification has been received, that the second bio-parameter measurement has been carried out, the second bio-parameter measurement being transmitted to the communication module.

The bio-parameter measurement system may comprise a second bio-parameter measurement device for providing the second bio-parameter measurement. The first and second bio-parameter measurement devices may be the same or separate devices.

The validation may comprise one or more from the group comprising: a confirmation received from the second bio-parameter measurement device; and, second measurement data relating to the second bio-parameter measurement from the second bio-parameter measurement device.

The first bio-parameter measurement device may be configured to perform the second bio-parameter measurement.

When the assessment module determines no second bio-parameter measurement is required on the basis of the first measurement data, the communication module may be configured to: notify a user that no further bio-parameter measurement is needed, or not transmit a notification to the user that a further bio-parameter measurement is needed.

Notifying a user that no further bio-parameter measurement is needed may further comprise providing a recommendation for the person.

Notifying the user that no further bio-parameter measurement is needed further comprises providing a health indicator for the person. The health indicator may be based on: the first bio-measurement data and the second bio-parameter measurement or the second bio-parameter measurement only.

The system may further comprise: at least one antenna for notifying the user via a wireless transmission.

The system may further comprise a user device for receiving the notification. The user device is a mobile phone. The user device may comprise a user interface having one or more input devices for inputting a validation response for transmission to the communication module as the validation, wherein the one or more input device comprises either or both of: a push button or a microphone for receiving a voice command.

One or more or all of: the first bio-parameter measurement device; the assessment module and the communication module may be provided within a single unit. The first and second bio-parameter measurement devices may be provided in the single unit.

The second bio-parameter measurement device may be hardwired or wirelessly connected to either or both of the communication module and the assessment module.

The system may further comprise at least one external connector for connecting the second bio-parameter device to either or both of the communication module and the assessment module.

The system may further comprise a central controller or server, wherein either or both the assessment module and communication module may be provided in the central controller or server.

The first bio-parameter measurement device may be battery powered.

The person and the user may be the same.

The first and second bio-parameter measurement devices may be the same device configured to obtain the first and second bio-parameter measurements. The first and second bio-parameter measurements may relate to the same bio-parameter. The first and second bio-parameter measurements may relate to different bio-parameters.

The second bio-parameter measurement may comprise a plurality of second bio-parameter measurements.

The system may further comprise a detector for detecting a presence or motion of a person in the vicinity of the first bio-parameter measurement device, wherein first bio-parameter measurement device is configured to carry out the first bio-parameter measurement following an output signal from the detector.

The first measurement data may comprise a plurality of first bio-parameter measurements. The first measurement data may comprise data from a plurality of different first bio-parameter measurements relating to different bio-parameters.

The first bio-parameter measurement device may comprise a temperature measurement device. The first bio-parameter measurement device comprise a camera for obtaining one or more images of the person in the visible spectrum. The first bio-parameter measurement device comprise an infrared detector. The first bio-parameter measurement device comprise a thermal imaging device. The first bio-parameter measurement device comprise an active reflected wave measuring device. The first bio-parameter measurement device may comprise one or more or any combination of the above.

The assessment module may be configured to determine a temperature of the person from the output signal of the thermal imaging device.

The assessment module may be configured to determine a heart rate of the person from the output signal of the thermal imaging device.

The assessment module may be configured to determine one or more of: a heart rate; respiratory rate and stress level from the camera.

The second bio-parameter measurement device may be configured to measure one or more or all of: temperature; heart rate; oxygen saturation; blood pressure; and blood glucose.

Notifying the user may comprise providing a visual indication comprising at least two notification states.

The visual indication may comprise a traffic light notification having three colours, each representative of different of either or both of the first measurement data or the second measurement data.

The communication module may be further configured to transmit one or both of the first measurement data and data relating to the second bio-parameter measurement to a third party.

Notifying the user may comprise transmitting a notification which results in one or more or all of: a visual, audible or haptic output.

The second bio-parameter measurement may require an action from the person.

Notifying the user that a second bio-parameter measurement is required ay comprise providing a prompt for the user.

In a second aspect, the present disclosure provides: a method of measuring a bio-parameter of a person comprising: obtain, using a first bio-parameter measurement device, first bio-parameter measurement data of the person without physical contact with the person; receive, at an assessment module, first measurement data from the first bio-parameter measurement device and determine whether a second bio-parameter measurement is required on the basis of the first measurement data; upon a positive determination that a second bio-parameter measurement is required, notify, using a communication module, a user that a second bio-parameter measurement is required; and, receiving, at the communication module, a validation that the user has undertaken the second bio-parameter measurement.

The validation may comprise: a confirmation received from the user.

The method may further comprise: obtaining, using a second bio-parameter measurement device, the second bio-parameter measurement.

The validation may comprise one or more from the group comprising: a confirmation received from the second bio-parameter measurement device; and, second measurement data relating to the second bio-parameter measurement from the second bio-parameter measurement device.

The first bio-parameter measurement device may perform the second bio-parameter measurement.

When the assessment module determines no second bio-parameter measurement is required on the basis of the first measurement data the method may further comprise: notifying, using the communication module, a user that no further bio-parameter measurement is needed, or, not notifying the user that a further bio-parameter measurement is needed.

Notifying a user that no further bio-parameter measurement is needed may further comprise a recommendation for the person.

Notifying the user that no further bio-parameter measurement is needed may further comprise providing a health indicator for the person. The health indicator may be based on: the first and second bio-parameter measurement or the second bio-parameter measurement only.

Notifying the user may comprise wirelessly transmitting a notification to the user.

The method may further comprise receiving, by a user device, the notification. The user device is a mobile phone.

The user device may comprise a user interface having one or more input devices for inputting a validation response for transmission to the communication module as the validation, wherein the one or more input device comprises either or both: of a push button or a microphone for receiving a voice command, the method further comprising:
receiving a user input via the one or more input devices.

The method may further comprise: connecting the second bio-parameter device to either or both of the communication module and the assessment module.

The person and the user may be the same.

The first and second bio-parameter measurement devices may be the same device configured to obtain the first and second bio-parameter measurements.

The first and second bio-parameter measurements relate to the same bio-parameter.

The first and second bio-parameter measurements may relate to different bio-parameters.

The second bio-parameter measurement may comprise a plurality of second bio-parameter measurements.

The method may further comprise detecting a presence or motion of the person in the vicinity of the first bio-parameter device and may carry out the first bio-parameter measurement following a detection of the presence of motion of the person.

The first measurement data may comprise a plurality of first bio-parameter measurements.

The first measurement data may comprise data from a plurality of different first bio-parameter measurements relating to different bio-parameters.

Obtaining the first bio-parameter measurement data may comprise obtaining a temperature measurement.

The method may further comprise: determining, using the assessment module, a temperature of the person using an output signal from a thermal imaging device.

The method may further comprise: determining, using the assessment module, a heart rate of the person from an output signal of a thermal imaging device.

The method may further comprise: determining, using the assessment module, one or more of: a heart rate; respiratory rate and stress level from a camera.

The second bio-parameter measurement may comprise one or more measurements taken from the group of: temperature; heart rate; oxygen saturation; blood pressure; and blood glucose wherein the second bio-parameter measurement device.

Notifying the user may comprise providing a visual indication comprising at least two notification states.

The visual indication may comprise a traffic light notification having three colours, each representative of different of either or both of the first measurement data or the second measurement data.

The method may further comprise: transmitting one or both of the first measurement data and data relating to the second bio-parameter measurement to a third party using the communication module.

Notifying the user may comprise providing one or more or all of: a visual, audible or haptic output.

The second bio-parameter measurement may require an action from the person.

Notifying the user that a second bio-parameter measurement is required comprises providing a prompt for the user.

In a third aspect, the present disclosure provides a computing device, comprising: a user interface and a processor configured to, upon receiving a notification from a bio-parameter measurement system as defined above, that a second bio-parameter measurement is required from a person, output one or more validation response options using the user interface for a user of the computing device to select and, upon receiving a selection of the one or more validation response options from the user, transmit the selected validation response to the bio-parameter measurement system.

The one or more validation response options may include one or more or all of: an acknowledgement that the notification has been received; an option to confirm that the required bio-parameter measurement has been carried out; an option to transmit the bio-parameter measurement. In some embodiments, the one or more validation response options include an option to confirm that the required bio-parameter measurement has been carried out. However, in other embodiments, it may be sufficient that the notification is acknowledged by a user, so that the system knows at least that the user is aware that a second bio-parameter measurement has been suggested to them. In yet other embodiments, the user may select whether to transmit the bio-parameter measurement to the communication module, or not to do so (for example if they deem that the result of second bio-parameter measurement is not of sufficient significance to warrant reporting).

The user interface may comprise one or more input devices for selecting the one or more validation response option, wherein the one or more input device comprises either or both of: a push button and a microphone for receiving a voice command.

The computing device may be a mobile phone.

In a fourth aspect, the present disclosure provides a method of validating a requirement for a bio-parametric measurement on a computing device comprising: receiving a notification from a bio-parameter measurement system that a bio-parameter measurement is required from a person, outputting one or more validation response options using a user interface for a user of the computing device to select and, upon receiving a selection of the one or more validation response options from the user, transmitting the selected validation response to the bio-parameter measurement system.

The one or more validation response options includes one or more or all of: an acknowledgement that the notification has been received; an option to confirm that the required bio-parameter measurement has been carried out; an option to transmit the bio-parameter measurement.

The user interface may comprise one or more input devices for selecting the one or more validation response option, wherein the one or more input device comprises either or both of: a push button and a microphone for receiving a voice command and the method further comprises: receiving the user selection via the one or more input devices.

In a fifth aspect, the present disclosure provides a non-transitory storage medium comprising electronic-processor readable code that, when read one or more electronic processors of a bio-parameter measurement system, configure the bio-parameter measurement system to carry out one or more of the methods of the present disclosure.

In a sixth aspect, the present disclosure provides a non-transitory storage medium comprising electronic-processor readable code that, when read one or more electronic-processors of a computing device, configure the computer device to carry out one or more of the methods disclosed herein.

The computing device may be a user computer device and may be mobile devices such as a mobile phone.

The skilled person will appreciate that except where mutually exclusive, a feature described in relation to any one of the aspects, examples or embodiments described herein may be applied to any other aspect, example, embodiment or feature. Further, the description of any aspect, example or feature may form part of or the entirety of an embodiment of the invention as defined by the claims. Any of the examples described herein may be an example which embodies the invention defined by the claims and thus an embodiment of the invention.

### BRIEF DESCRIPTION OF THE SEVERAL VIEWS OF THE DRAWINGS

The invention will now be described, by way of example only, with reference to the accompanying drawings, in which:

FIG. 1 shows a schematic representation of a bio-parameter measuring system;

FIG. 2 shows a flow diagram of a method for obtaining a bio-parameter measurement;

FIG. 3 shows a schematic representation of a bio-parameter measuring system;

FIG. 4 shows a schematic representation of another alternative bio-parameter measuring system;

FIG. 5 shows a user device which may be used with the bio-parameter measuring system.

### DETAILED DESCRIPTION OF THE EMBODIMENTS

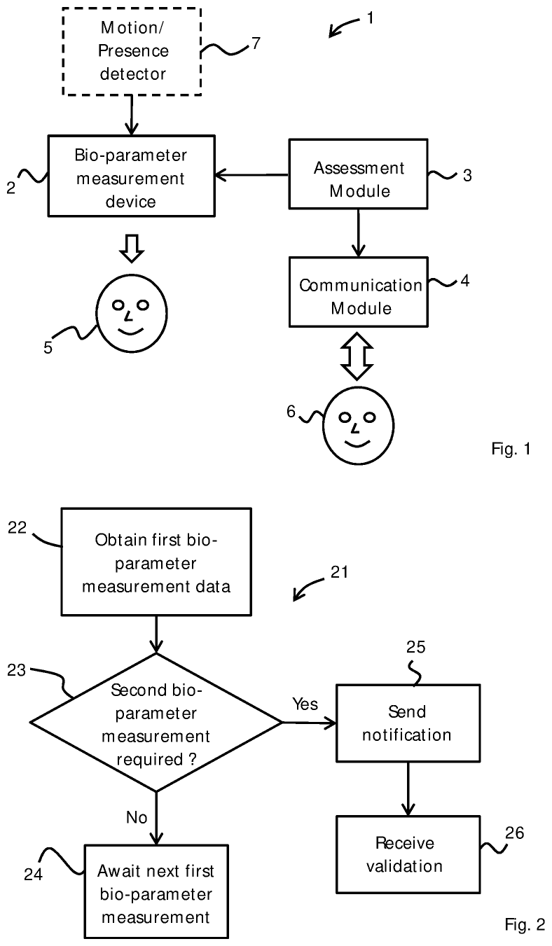

FIG. 1 shows a bio-parameter measuring system 1 comprising a first bio-parameter measurement device 2 for determining a bio-parameter of a person 5; an assessment module 3 configured to receive first measurement data from the first bio-parameter measurement device 2 and determine whether a second bio-parameter measurement is required on the basis of the first measurement data.

The system 1 also comprises a communication module 4 configured to: upon a positive determination that a second bio-parameter measurement is required, transmit a notification to the user 6 in which the notification is indicative that a second bio-parameter measurement is required. The communication module 4 may also be configured to receive a validation that the user has received the notification or undertaken the second bio-parameter measurement.

The systems and methods described herein provide an improved way to obtain bio-parameter measurements without physical contact from a person except where necessary. Receiving a validation from a user confirming that the second test has been carried out or measurement data from a subsequent bio-parameter measurement allows for improved remote monitoring of a person by a user. In the case of remote monitoring the user may be a parent, carer or a medical professional, however, it will be appreciated that, in some embodiments, the person being measured and the user may be the same. The systems and methods described herein allow symptoms of medical conditions to be monitored and diagnosed more readily and the tracking of a person's health to monitor in an unobtrusive way.

The first bio-parameter measurement device 2 may be any suitable device known in the art for obtaining a bio-parameter from the person 5. In some embodiments, the first bio-parameter measurement device 2 may be a non-contact device which allows the first measurement data to be obtained without physical contact from the user. Hence, the first bio-parameter measurement device may not require any purposeful interaction from the person 5. As such, the first bio-parameter measurement device 2 may be configured to operate in the background, possibly on an on-going or scheduled basis, such that the person 5 is not disturbed or inconvenienced in any way when the bio-parameter measurement is made. As such, the system is less obtrusive for a person 5 and may provide more regular and reliable measurement data acquisition for the person 5.

The term without physical contact may be understood to mean that the first bio-parameter measurement device may obtain measurement data whilst physically separated from the person so as to not be in direct physical contact. The first bio-parameter measurement device may be a wave-based measurement device which receives measurement data, or information pertaining to the measurement data, via electromagnetic waves (visible light, infrared, microwave or radio waves, for example) or acoustic waves. Thus, the bio-parameter measurement may be based on waves received from the person over the air. The electromagnetic waves may be infrared light or visible light, for example. The waves may originate from the person (e.g. black body TR radiation), be scattered from the person (e.g. visual images) or be reflections from the person (e.g. a measurement based on reflected waves, e.g. radar, sonar, or lidar).

The first bio-parameter measurement device 2, may be configured to constantly capture first measurement data from a person 5 whilst the person is at a suitable location for the measurement to be acquired. However, in some embodiments the first bio-parameter measurement device 2 may be controlled so as to be operable during specific times or following one or more specific triggers. The specific times may be in accordance with a predetermined schedule, for example, in accordance with a daily routine, or additionally or alternatively, when the presence or motion of a person is detected. Limiting the times at which the first bio-parameter measurement device 2 is configured to be operable can save power. This is particularly advantageous where the first bio-parameter measurement device 2 is a single unit with self-contained power source such as a battery.

The first bio-parameter measurement device 2 may be configured to measure one or more bio-parameters from the group comprising: temperature, heart rate; respiratory rate; oxygen saturation, blood pressure and stress. The stress may be characterised as mental stress and based upon Baevsky or US/European Index level measurements. Stress may be determined on the basis of a Heart Rate Variability measurement.

The first bio measurement device 2 may comprise one or more bio-parameter sensing or detection devices such that one or more bio-parameters may be obtained as part of the first measurement data. The one or more bio-parameter sensing or detection devices may be referred to as sub-devices of the first bio-parameter measurement device 2. Each of the first bio-parameter measurement sub-devices may be non-contact and/or non-interactive.

In some embodiments, the first bio measurement device 2 may be considered to be a temperature measurement device for obtaining the temperature of the person. The temperature measurement device may be any suitable device known in the art and may provide an output signal which is indicative of the person's temperature. The temperature measurement device may include one or more temperature sensors and a temperature determination module. The temperature determination module may receive and process an output signal from the or each temperature sensor to provide a temperature reading. The reading may be the temperature measurement, or multiple readings may be averaged over a short period time (e.g. 1-2 seconds) to provide the temperature measurement. The temperature measurement device may comprise a plurality of individual temperature sensors. The temperature sensor may comprise a thermopile or in the case of a plurality of temperature sensors may comprise an array of thermopiles. The first bio-parameter measurement device may include a temperature measurement device and may include multiple detectors or sensors to measure temperature and one or more other bio-parameters. The temperature measurement device may also comprise one or more thermal cameras, as described below.

The first bio-parameter measurement device 2 may comprise one or more from the group consisting of: a camera for obtaining one or images of the person in the visible light spectrum; an infrared detector; a thermal imaging device; and, an active reflected wave measuring device. In some embodiments, each or any of this group may be used to perform a bio-parameter measurement, However, in other embodiments, one or more of this group might not perform a bio-parameter measurement and this additionally or alternatively may be included to have a function that is independent of the first bio-parameter device, but might be used to assist in acquiring the first measurement data or second measurement data (described elsewhere herein) in cases where the second measurement data is performed by the first bio-parameter measurement device 2. For example, a visible light camera may be used to identify a suitable area of a person from which the measurement may be taken such as the forehead for a temperature measurement or the chest for a respiratory rate. This data may be used in conjunction with other detectors or sensors such as a thermal imaging device or active reflected wave measuring device to obtain the bio-parameter measurement. It will be appreciated that obtaining one or more images with the first bio-parameter measurement device may not be a bio-parameter measurement per se, but such images may be analysed to extract bio-parameter information assist with determining the first measurement data.

As an additional or alternative example, a camera associated with the first bio-parameter measurement device 2 may be used for facial recognition to determine who the person is, for example, for providing a personal measurement and data tracking purposes. In some embodiments, one or more of the group of first bio-parameter measurement sub-devices may be used to obtain location information relating to the person and a bio-parameter measurement.

The active reflected wave measuring device may comprise any device in which sound or electromagnetic waves are transmitted to the person and reflected waves received and processed to determine the presence or position of the person and possibly locate one or more features and/or measure one or more bio-parameters such as a respiration rate. The active reflected wave measuring device may comprise any kind of one, two or three dimensional locator which may, for example, be based on one or more of: SONAR, RADAR or LIDAR, but in some embodiment, more particularly, RADAR or SONAR, which may be cheaper than LIDAR.

The bio-parameter measuring system 1 may comprise one or more detectors 7 for detecting the motion or position of a person. The detector 7 is optional as represented by the dashed line. The detector 7 for detecting the presence or motion of a person may be used to control bio-parameter measuring device 2 such that the bio-parameter measuring device 2 is operable only when a person is in the correct location to have the measurement taken. Hence, when a signal is received from the detector 7, the bio-parameter measurement device 2 may be configured to acquire the bio-parameter from the person 5. When the presence of motion of a person is not detected, the bio-parameter measurement device 2 may be in a low-power/powered-down state (e.g. a sleep or off state) to conserve power.

Presence detectors are well known in the art. The presence detector may be a detector configured to determine the presence of a person at a location. The state or output of a presence detector may change when a presence of a person changes from present to not present or vice versa.

Motion detectors are well known in the art. The motion detector may be a detector configured to determine the motion of a person at a location by using any suitable motion detection sensors. The state or output of a motion detector may change each time motion of a person is detected.

The motion detector may be a passive infrared (PIR) detector or a Doppler-based motion detector, which may be based on ultrasonic sound waves, for example. The sensor of the passive infrared detector may be a pyroelectric sensor which typically results in particularly energy efficient PIR detector.

Presence detectors may comprise an active light (e.g. infrared) detector (for example based on a pair of devices in the form of an LED and a photodiode, which may be based on a detected reflection for which the LED and photodiode may be collocated, or based on the interruption of a light path (e.g. detecting whether an object passes between an LED and photodiode).

The presence detector may additionally or alternatively comprise a proximity sensor, as known in the art.

The active reflected wave measuring device described above may be used as the detector 7.

The first measurement data may be any data suitable for assessing whether a further bio-parameter measurement is needed. The first measurement data may be understood to be an output signal from one or more sensors or detectors provided as part of the first bio-parameter measurement device 2. Additionally or alternatively, the first measurement data may comprise the output from an intermediary module which receives the output signal from a sensor or detector and determines a measurement of interest, such as a temperature, from the output signal.

The first measurement data may comprise data from a plurality of a first bio-parameter measurement sub-devices, detectors or sensors. The first measurement data may relate to one or more bio-parameters which, in combination, provide the indication that a second bio-parameter measurement is required.

For each measured bio-parameter, the first measurement data may comprise a single value or range of values. In some embodiments, the first measurement data may comprise a single measurement such as a temperature as measured by, for example, an IR detector. In other embodiments, the first measurement data may comprise an average temperature. In the case of a range of values, the first measurement data may, for example, be measurement data taken from a series of sequential measurements.

The second bio-parameter measurement device (not shown in FIG. 1) may be configured to obtain a second bio-parameter measurement following the determination that a further bio-parameter measurement is required. The second bio-parameter measurement device may be configured to obtain second measurement data or a plurality of second measurement data. As with the first measurement data, the second measurement data may comprise measurement data relating to a plurality of different bio-parameters.

The second bio-parameter measurement device may comprise one or more devices for measuring one or more from the group comprising: heart rate; oxygen saturation; blood pressure; and blood glucose. Suitable devices for measuring these bio-parameters are known in the art.

In some embodiments the second bio-parameter measurement device may be a separate bio-parameter measurement device. The second bio-parameter measurement device may be separate to the first bio-parameter measurement device 2 and located remotely from the first bio-parameter measurement device 2. The second bio-parameter measurement device may be connected to the assessment module 3 via a wireless connection or a hardwired connection. The second bio-parameter measurement device may be handheld and/or require contact with the person 5 to enable a measurement to be obtained. The second bio-parameter may comprise a user device in the form of a mobile device, e.g. a mobile phone or a wearable device.

In some embodiments, the second measurement data may relate to the same type of bio-parameter as the first bio-parameter measurement or a different bio-parameter. For example, when the first measurement data relates to a temperature of the person 5, the second measurement data may relate to the body temperature measured using a second bio-parameter measurement device, or using the first bio-parameter measurement device 2 in an interactive way. More specifically, in some embodiments the first bio-parameter may be a temperature taken from a person's forehead so as to be indicative of a person's core body temperature. However, depending on the type of measurement device or technique used, the accuracy of the forehead temperature measurement may be inaccurate and may need to be verified. In this case, the person's temperature may be acquired a second time using either the same first bio-parameter measurement device used in an interactive way, such as by instructing the person to open their mouth such that an oral temperature can be obtained, or a second bio-parameter measurement device which takes the measurement in a different way, for example by inserting a probe into the persons ear or the like. Other variants on the types of temperature measurement devices and techniques will be known to the skilled person.

In some embodiments, the first bio-parameter measurement data may be sufficient to indicate that a different type of bio-parameter is required, such as one or more of a blood pressure or blood glucose level. The different types of bio-parameter may be selected on the basis of the person's medical history, for example.

The first and/or second bio-parameter measurement devices may use photoplethysmography, PPG, to obtain the first and/or second measurement data based on camera-captured images, as known in the art. The second bio-parameter measurement device may comprise a pulse oximeter. Additionally or alternatively, the first and second bio-parameter measurement devices may use remote photoplethysmography, rPPG, in which visible light images of the person are used to determine characteristics such as heart rate, heart rate variability, blood pressure, oxygen saturation, respiration rate, temperature, mental stress or physiological stress, as known in the art. See for example, Sinhal et al, “Estimating vital signs through non-contact video-based approaches: A survey”, 2017 International Conference on Recent Innovations in Signal processing and Embedded Systems (RISE), 27-29 Oct. 2017, and BVue Visual Vitals smartphone app provided by Binah.ai, www(dot)binah(dot)ai. The camera may be provided as part of the first or second bio-parameter measuring device. In one embodiment, a user device may incorporate the camera which may be used to capture one or more visible light images for rPPG processing and analysis. The rPPG analysis may be carried out on the mobile device or remotely, depending on the amount of computation required.

The second bio-parameter measurement device may comprise any known ECG electrodes or a blood pressure monitor or sensors.

The assessment module 3 may be configured to receive first measurement data from the first bio-parameter measurement device 2. The assessment module 3 may receive the first measurement data by any suitable means of communication.

The first bio-parameter measurement device 2 and the assessment module 3 may be hardwired or wirelessly connected such that the first measurement data may be transmitted from the first bio-parameter measurement device 2 to the assessment module 3. For example, the assessment module 3 may be housed within a single unit and receive the first measurement data directly from the first bio-parameter measurement device 2. In other examples, the assessment module 3 may be housed within a separate unit or be provided by a different entity such as a server which is connected to via a network.

The assessment module 3 may be configured to determine whether a second bio-parameter measurement is required on the basis of the first measurement data obtained by the first bio-parameter measurement device 2. This assessment may comprise a comparison of the first measurement data with an expected value or range of values. Where the first measurement data is a value or range of values within acceptable limits, then the assessment module 3 may determine that no further second bio-parameter measurement is required in response to the first measurement data. In the alternative, when the first measurement data is not a value or range of values within acceptable limits then the assessment module may make a positive determination that a second bio-parameter measurement is required.

The expected value or range of values against which the first measurement data is compared will typically be determined on the basis of medically acceptable values or ranges. The range of values may comprise an upper limit and a lower limit. The upper and lower limits may bound and define an acceptable range.

In some embodiments, the first bio-parameter measurement may be a temperature of a person 5. The medically acceptable temperature range may, if accurately measured, be between 36.1° C. to 37.2° C. Hence, a temperature falling below the upper limit, above the lower limit or between the upper and lower limits may be determined to be acceptable. For example, a positive determination for a second temperature measurement may be made when a person's temperature is over the upper limit which would be indicative of a fever.

In addition or as an alternative to the above, it will be appreciated that the acceptable range may be bounded by other upper and lower limits or by more than one lower limit or more than one upper limit. For example, a person's temperature maybe classified as a low grade temperature or low grade fever if above a first upper limit of 37.2° C. but below a second upper limit of 38° C., and a high-grade temperature or temperature if above 39° C. Hence, there may be multiple limits which result in different responses from the bio-parameter measuring system.

In some embodiments, a temperature above 38° C. may in some embodiments trigger in a second bio-parameter measurement as this temperature may be indicative of a fever. In particular, if the person's skin temperature is not misleadingly inflated above their core temperature, for example due to a recent exposure to a hot environment, then it may be assumed that the person has a fever.

If the temperature is between 37-38° C., then in some embodiments a second bio-parameter measurement may be triggered since it is particularly unclear whether the person does or does not have a fever. If the temperature is between 32-37° C. the measurement may be taken as not being indicative of a fever and so in some embodiments no further bio-parameters are triggered. If the temperature is less than a lower limit, for example, 32° C., it may be indicative that there is a fault with the measurement system or the first measurement data has not been captured correctly. Therefore, in some embodiments, a second bio-parameter measurement may be triggered in this scenario too, and/or a fault notification may be issued.

As described below, the output of the assessment unit 3 may be provided on a visual display. The visual display may comprise one or more colour outputs. The colours may correspond to, for example, a red display if the temperature is indicative of a fever, e.g. >38° C., a yellow display if the temperature may be indicative of a possible fever, e.g. 37-38° C., a green light if the temperature is indicative of no fever, e.g. 32-37° C., and a flashing display or different colour if the temperature appears to be erroneous, e.g. <32° C. It will be appreciated that other visual displays may be used, including alternative colours to those described here.

It will be appreciated that the values and one or more limits referred to above may be referred to as thresholds or threshold values.

It will be appreciated that the assessment module 3 may comprise or have access to one or more databases which comprise the acceptable limits or range of values upon which a determination for a second bio-parameter measurement may be required.

Following a positive determination that a second bio-parameter measurement is required, the bio-parameter measuring system 1 may be configured to transmit a notification to a user (e.g. via the communication module 4). The notification may be indicative that a second bio-parameter measurement is required.

The purpose of the second bio-parameter measurement is to determine any significant underlying health concerns that may require further action to be taken. The second measurement data may be used to determine one or more symptoms more accurately, or gather a broader range of measurement data which may aid with diagnosing a possible health status, underlying health condition or illness by a remote medical professional.

The person 5 may be any person for whom bio-parameter measurements would be usefully obtained. The person 5 may be a resident or occupant of a premises. The premises may be a dwelling or other building which a person is regularly located in and which provides a suitable location for obtaining bio-parameter measurements.

In some embodiments the user 6 may be the person 5 from whom the first bio-parameter measurement has been taken. In other embodiments the user 6 may be a different person. The user 6 may be, for example, a parent or guardian of the person 5 being monitored or a carer responsible for the health of the person 5 being measured. In other embodiments, the user 6 may be a remote medical professional. Other examples may exist.

The bio-parameter measuring system, and more particularly, the communication module, may be configured to notify a user 6 that a second bio-parameter measurement is required, and/or, notify a user 6 that a second bio-parameter is not required. As such, the communication module may be configured to provide one or more notifications. The one or more notifications may provide information relating to the first measurement data or to the second measurement data. Such a notification may be for one or more of the group comprising: updating the user 6 that a bio-parameter measurement has been performed; providing results of the bio-parameter measurement; a recommendation relating to a bio-parameter measurement; providing a health indicator regarding the person 5; indicating that a further bio-parameter measurement is required; indicating that a further bio-parameter measurement is not required.

The notification may be provided as an indication on a user interface of a user device. The user interface may comprise one or more or all of: a haptic, visual or audible output via which the notification can be provided to a user 6. In some embodiments, the user interface may incorporate a visual display comprising one or more illuminated display elements. The user interface may be provided as part of a user device, or may be provided as part of the first or second bio-parameter measurement device or a housing thereof. In other embodiments, the user interface may comprise one or more speakers through which an audible alarm may be sounded or spoken words issued.

As well as providing an output for conveying the notification to the user 6, the user interface may additionally provide one or more input devices for allowing a user to input a validation response to the notification.

In some embodiments, the notification is provided to a user device comprising the user interface. The user device may be any computing device such as a desktop computer, workstation or a mobile device such a mobile phone. The user device may comprise a computer program (e.g. an application) which, upon receipt of a notification, displays the notification to the user, together with visual or audible prompts as to how to respond. For example, the user device may comprise a display on which the notification can be presented to the user 6 or a speaker which can provide an audible alert to the user 6 or spoken words. In the case of a touch screen display, the display may also be configured to provide the user 6 with one or more virtual buttons with which the user 6 can provide a validation response.

In other embodiments, the first or second bio-parameter measurement device may comprise one or more display panels comprising a pixelated display and/or one or more LEDs which are illuminated according to predetermined colours or display timings (such as a solid or flashing) to convey that the user 6 is required to take some further action in response to the first measurement data.

In addition to providing a notification in regard to the first measurement data and the requirement for a further bio-parameter measurement, further notification may comprise one or more taken from the group comprising: a recommendation, for example, to stay at home for the day (for example, where the person has a confirmed low grade fever), to visit a doctor (for example, in the event that the first or second bio-parameter measurements indicate a bio-parameter of serious concern, such as a confirmed high temperature or high blood pressure) or to recommend a further bio-parameter measurement be taken at a predetermined later time. For example, if a person is shown to have a temperature at the high end of the normal range, it may be something to monitor over the coming days rather than requiring an immediate interactive further bio-parameter measurement. For example, an elevated temperature may relate to recent physical activity or the person having stepped inside from outside on a hot day. In such a situation, it may be sufficient to retest the person at a later time.

The health indicator may comprise the recommendation or maybe a separate communication. The health indicator may be a visual indication or audible indication and may be provided as a spoken word along the lines of “you appear to be fine today” or “we have not detected any abnormalities in your bio-parameters this morning”. A visual indication may be a written message on an LCD display such as “You're temperature is within normal limits. Have a nice day!”, or a colour coded output in which predetermined colours or display patterns correspond to different health conditions. For example, a user interface comprising an output display may include one or more LEDs of varying colours in which green corresponds to a clean health check and red corresponds to a requirement for having further measurements carried out. Alternatively, an LED may be configured to provide a solid light when everything is within normal parameters or a flashing light when further tests are required. In some embodiments, a different light or flashing light may be used to provide as a notification that there is a possible fault with the system or the measurement appears to be erroneous.

In some embodiments the recommendation and/or health indication will be provided in response to the first and second measurement data or the second measurement data only. Hence, the recommendation and/or health indicator may be dependent on the second measurement data only, particularly when the result of the first bio-parameter measurement either results in a requirement for a further bio-parameter measurement or no action.

A notification which indicates that a second bio-parameter measurement is required may be referred to as a first notification, a first notification type or a further test notification. A notification which indicates that a second bio-parameter measurement is not required may be referred to as a second notification, a second notification type or a no further test notification. The communication module may be configured to transmit the first notification type or the second notification type, depending on the first measurement data and a requirement for second measurement data. It will be appreciated that in some embodiments no notification may be sent if no further test is required.

Hence, in some embodiments there may be a communication module configured to transmit a notification to a user. The notification may be indicative that a second bio-parameter measurement is required upon a determination that a second bio-parameter is required or may be indicative that a second bio-parameter measurement is not required upon a determination that a second bio-parameter is not required. Either notification may be comprise a visual indication for display for the user. The visual indication may comprise one or more colours in which the colours are indicative of the type of notification.

Upon receiving the notification a user 6 can provide a validation to the system in relation to the notification and, where applicable, the requirement for a further bio-parameter measurement. The validation may comprise one or more from the group comprising: a confirmatory acknowledgement that a further bio-parameter measurement is required; a confirmation that the further bio-parameter measurement has been carried out; a confirmation that the further bio-parameter measurement has been carried out by providing the results of the further bio-parameter measurement. The validation may be received from a user 6 e.g. from a user device, or may be provided by the second bio-parameter measurement device.

In some embodiments, the validation may be provided by a user 6 via a user device such as a mobile phone. The user device may be configured to receive a voice response in relation to the notification and/or may be configured to present a user selectable validation response option to a user on a user interface. For example, the user interface of a user device may comprise one or more virtual buttons which a user can select. Selection of the one or more buttons may result in the transmission of the selected validation response.

As noted above, the notification may be sent to a user 6 and the user 6 may or may not be the person 5 whose measurement has been taken. Where the user 6 is not the person 5 being measured, the possible validation responses may be different to those provided when the user is the person 5 being measured. For example, when the user 6 is not the person 5 being measured the validation response may relate to whether the user 6 has located the person 5 being measured and/or whether the person 5 has carried out the second bio-parameter measurement.

In some embodiments, where the user 6 and person 5 are not the same, the notification may be sent to the user 6 and the person 5 being measured.

The preceding description generally relates to the transmission of a notification when a positive determination for the requirement of a second bio-parameter measurement is made. When a negative determination is made and no further bio-parameter measurements are deemed necessary at that time, the bio-parameter measuring system 1 may be configured to provide no notification or transmit a notification indicative that no further bio-parameter measurement is required. In the event that no further measurements are required, the notification may be provided as a LED on the housing of the first bio-parameter measurement device, as noted above in relation to the health indicator.

In the event that no validation is received, the bio-parameter measurement system 1 may send a further notification to another user. The other user may be a parent or guardian, carer or medical professional. Alternatively or additionally, the system may send a further notification to the user. The further notification may be a repeat of the original notification or may include a selectable option to ignore the notification and requirement for a further bio-parameter measurement.

The bio-parameter measuring system may form part of a medical monitoring system in which measurement data from one or more bio-parameter measurement devices is used to provide remote medical monitoring of people. Bio-parameter measurements may be shared with one or more medical professionals in order to monitor the health of the person being monitored in a relatively unobtrusive manner. This may be advantageous for recording historical data for a patient and also detecting the onset of any underlying health conditions or illnesses.

The bio-parameter measuring system 1 may be housed within a single unit or may be a distributed system in which different aspects are carried out in different entities. In the case of the bio-parameter measuring system being housed within a single unit, the unit may be located at a fixed location within a premises and configured to obtain the first measurement data from the person without physical contact from the person.

The single unit may comprise any combination of the group comprising: first and second bio-parameter measurement devices; the assessment module 3 and the communication module 4.

The location of the first bio-parameter measurement device 2 may be an area in front of a mirror in a bathroom where a person is likely to regularly stand and face the first bio-parameter measurement device 2, thus enabling regularly bio-parameter measurements in a non-contact and/or non-interactive way to be reliably acquired. In other examples, the bio-parameter measurement device 2 may be in a person's bed or adjacent to a lavatory or sink (in a bathroom or kitchen). Other exemplary locations are within the scope of the disclosure. The first bio-parameter measurement device 2 may be mounted at or above (optionally no more than 50 cm above) head level. By being mounted at such a height, the first bio-parameter measurement device 2 may be in an ideal position to measure forehead temperature when a person is standing in front of the device. The person 5 may in such cases be relatively close to the device, e.g. within 2 meters. Thus, the first bio-parameter measurement device 2 may be mounted at 180 cm or higher (but optionally not higher than 220 cm) in some embodiments.

FIG. 2 shows a method 21 of undertaking a health check using a bio-parameter measuring system 1. The bio-parameter measuring system 1 may be the system described in FIG. 1 but other systems may be used where appropriate. The method steps 22 to 26 may be carried out with reference to the features discussed in connection with FIG. 1 which will not be repeated here for brevity.

At step 22, first measurement data is obtained. The first measurement data may be obtained by measuring a bio-parameter of a person 5 using a first bio-parameter measurement device 2. Once the first measurement data has been obtained, a determination may be made at step 23 as to whether a second bio-parameter measurement is required on the basis of the first measurement data. Where a positive determination is made that a second bio-parameter measurement is required, a notification may be transmitted to a user (step 25). The notification may be indicative that a second bio-parameter measurement is required. Following this, a validation may be received, step 26, that the user has undertaken the second bio-parameter measurement.

If there is a negative determination at step 23 and no further bio-parameters are required at that time, the system may take no further action and await the next first measurement data, thereby returning to step 22. Alternatively, the system 1 may be configured to send a notification to a user 6, providing an indication that the first measurement data was acceptable and no further bio-parameters are required. Such a notification may comprise a general indication of good health, or that no poor health was detected, rather than a specific reference to any particular measurement data, as per the health indicator discussed above.

The first and second bio-parameter measurements may be a first stage referring to the first bio-parameter measurement, and a second stage referring to the second bio-parameter measurement. The first stage measurements may relate to non-contact and/or non-interactive measurements in which a person 5 is subject to one or more bio-parameter measurements without actively engaging with or interacting with the bio-parameter measurement device. The second stage measurement may relate to interactive bio-parameter measurements which require some engagement from the person being measured. The second bio-parameter measurements may require a person to maintain a position or pose whilst the measurement is carried out or may require one or more actions to be followed. Additionally or alternatively, the second bio-parameter measurement may require some form of physical contact between the person 5 and the second bio-parameter measurement device.

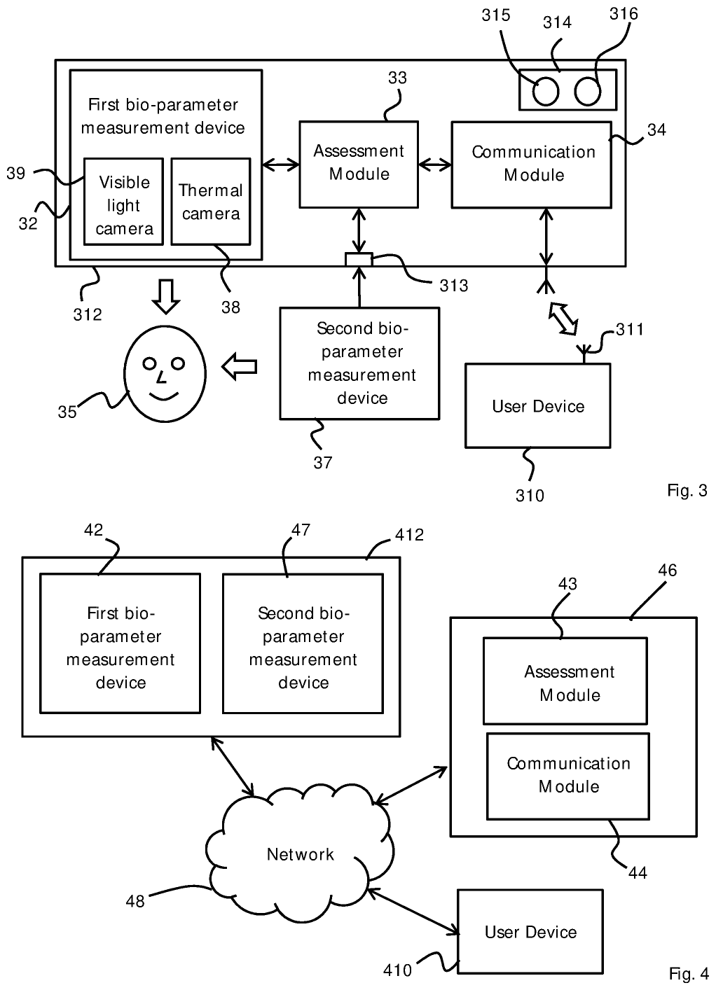

FIG. 3 shows an embodiment of a bio-parameter measuring system 31 comprising a first bio-parameter measurement device 32, an assessment module 33, a communication module 34, a second bio-parameter measurement device 37, and a user device 310. The first 32 and second 37 bio-parameter measurement devices may be configured to obtain a bio-parameter measurement, i.e. first and second measurement data, from the person 35. Many features of the embodiment of FIG. 3 have been described in connection with the corresponding elements of FIG. 1, and these may not be repeated below for the sake of brevity.

In the embodiment of FIG. 3, the first bio-parameter measurement device 32 comprises a thermal camera 38 and therefore a thermal imaging sensor. Thus, the first bio-parameter measurement device 32 may comprise a plurality of sensors in the form of respective pixels and a processor configured to determine the temperature of a person from an image captured with the thermal imaging device 38. In some embodiments, a temperature measurement may be determined for each pixel. Determining a temperature of the person may comprise identifying a region within a field of view of the thermal camera 38, wherein the region is represented by a subset of the pixels that is determined to represent a person's core temperature. For example, the region may correspond to a region for which a group of pixels have similar values to each other (to negate error/noisy pixels) which define a maximum temperature region within the field of view or within a portion of the field of view. Additionally, or alternatively, the region may be limited to a region corresponding to a person's forehead, for example based on image recognition. The temperature of the person may be calculated from an average of the measured temperatures within the region.

In addition to a thermal camera the first bio-parameter measurement device 32 may also comprise a camera 39. The camera may be a visible light camera for capturing one or more images.

The camera 39 may capture one or more image(s) that correspond to (e.g. simultaneously or substantially simultaneously with the capturing of) thermal image(s) using the thermal camera 38 (or some other form of temperature measurement device). Image processing may be performed on the image(s) from the camera 39 to confirm that a person is present and/or to identify a location that corresponds to a particular part of the person that is of interest, for example their forehead. The location may be correlated with the corresponding thermal image to identify (or improve identification of) a part of the thermal image that corresponds to that same region/part of the person 35. For example the camera 39 and the thermal camera 38 may have the overlapping, or the same, fields of view. The use of the camera 39 in such a manner may provide better feature (e.g. forehead) recognition than a thermal camera 38 because a such a camera 39 may have a higher resolution than the thermal camera 38 which may allow for improved feature recognition.

Generally throughout this disclosure, it is to be understood that reference to the temperature of a person may be taken to mean a temperature associated with a person and may or may not refer to the person's core temperature. As such, the temperature of a person may be a temperature indicative of or correlated with the core temperature. In particular, the temperature measurement device may measure a surface temperature of a person. The surface temperature may be taken from an area of skin, for example, the forehead as noted above, but could also be elsewhere such as inside their mouth when presented to the temperature measurement device. An oral temperature measurement may provide an improved temperature measurement which is more accurate and reliable and more indicative of a core body temperature. The methods described above to identify the forehead may likewise be used to identify an inside of an open mouth. The temperature measurement of an area of skin with the camera 38 and thermal camera 39 may be considered to be a first bio-parameter measurement as it can occur passively without physical contact or interaction from the person 35. The example of an oral temperature may be considered to be a second bio-parameter measurement because it relies on an action from the person. The action may be in response to a prompt or instruction provided by the notification. That is, the person is required to follow one or more actions, such as opening their mouths and standing in a particular location in a particular orientation or pose. Other example prompts may include an instruction of what measurement to take, how to take the measurement, and when to take the measurement.

In the case of a temperature measurement, it will be appreciated that the ambient environment in the location of the first bio-parameter measurement device 2 may affect the surface or skin temperature of a person, as might the activity of the person immediately prior to the temperature measurement. For example, possible causes of error in temperature measurement from the skin versus the core temperature may result from the person being outdoors on a hot (or cold) day or having just exercised. If a person has just been in a hot sunny environment their skin temperature relative to their core temperature may be high. Conversely, their skin temperature relative to their core temperature may be low. This can be countered (even if only partially) by having the device in a suitable location. The location may be temperature controlled and/or relate to an area of the house which is less likely to be entered immediately following entering the house.

Further, taking a temperature before a certain time of day (likely before they have left the house), and in some embodiments, only at that time, may reduce such external influences.

When the system is configured to measure the temperature of a person, the temperature of other objects may be measured. However, the system may be configured to ensure, or at least encourage, that the temperature of a person is captured even where other items or animals may also be measured. This may be achieved for example by using the motion/presence detector minimise or eliminate confusion with people and other objects, using known methods. Further, the assessment unit may be configured to provide a positive determination for temperatures which are within predetermined ranges.

It will be appreciated that other first bio-parameter measurement devices 32 may be used in place of or in addition to the thermal camera 39 and the visible light camera 38. For example, the visible light camera 38 may be used to determine one or more from the group comprising: respiratory rate, oxygen saturation, heart rate and stress, with or without a temperature measurement. Further, the temperature measurement may be obtained with an IR detector comprising one or more thermopiles, for example.

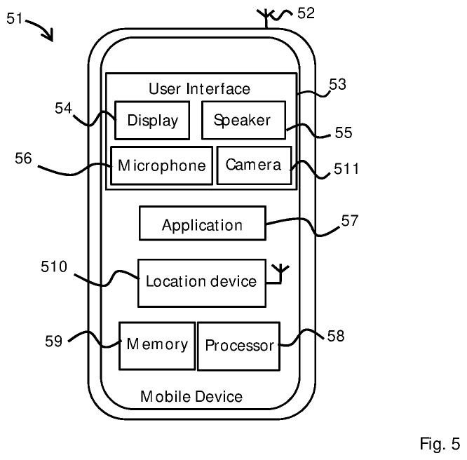

The user 6 of FIG. 1 is referred to here by reference to a user device 310. The user device 310 may be any device which the user, i.e. the recipient of a notification from the communications module 34, may receive the notification on. Typically, the user device 310 will comprise a computing device having at least one visual display, an audio output device, e.g. a speaker and an audio input device, e.g. a microphone. Further details of a user device may be found in relation to FIG. 5.

The user device 310 may comprise an antenna 311 which is configured to receive and/or transmit data from or to the communications module 34. The connection between the communication module 34 and the user device 310 will be wireless and achieved using conventional wireless transmission technology. Thus, the user device 38 may communicate with the communication module 34 via the internet, a wide area network, a wireless local area network, or short range wireless communications using known standards and/or protocols. The transmission may be via a short to medium range communication protocol, e.g. WiFi®, Bluetooth® (including lower power Bluetooth®), IEEE 802.15.4, or via a cellular communication protocol (e.g. 3G, 4G, 5G) to a server; or directly to a mobile phone (e.g. Bluetooth®).

The notification may be provided as a drop down notification as known in the art and/or may prompt the user 6 to open an app on their user device 38 such that the notification can be conveyed and a validation response initiated.

FIG. 3 also shows a housing 312 in which the first bio-parameter measurement device 32, assessment module 33 and communications module 34 are housed. As such, these components can be considered to provide a single unit. The housing 312 may comprise any suitable container as known in the art such as a moulded plastic housing. The housing 312 may be mounted (or mountable) to a flat surface such as a mirror or a wall such that it can be provided in a fixed location within a premises. The mounting may be provided by any suitable means such as a wall bracket, adhesive patches such as an adhesive sticker or tape, or by screwing or bolting the housing directly to the wall. Hence, the housing may comprise one or more features which aid the wall mounting, such as: a one or more holes which are accessible to receive a fixture such as a screw; an adhesion zone for receiving one or more adhesive patches; and a bracket for fixing to the wall. In use, the housing may be provided at a suitable location within a premises.

In the example of FIG. 3, the second bio-parameter measurement device 37 is located external to the housing 312. The second bio-parameter measurement device 37 may be a dedicated piece of equipment solely for measuring one or more specific bio-parameters, or may be a multifunctional parameter measuring device such as a mobile phone with a suitable camera, or other sensors, and appropriate software for deriving the second measurement data from the camera and/or sensors using rPPG techniques, for example.

The second bio-parameter measurement device 37 may be wirelessly connected to the communications module 34 or assessment module 33. However, as shown in FIG. 3, the connection may also be provided by a hardwired connection via an external connector 313. The external connector 313 may be any suitable connector such as a USB or USB-C, for example. Hence, the connection between the second bio-parameter device 37 and assessment module 33 and/or communication module 34, may be a temporary hardwired connection. The connection may be made simply to download the second measurement data by way of providing the validation, as described above. In some embodiments the second bio-parameter sensor 37 may be hardwired via a network connection within the premises.

The housing 312 may comprises one or more user interfaces in the form of a visual display 314. The visual display 314 may comprise a pixelated screen for displaying images or text, or may simply be provided with one or more LEDs 315, 316 which can be used to convey the notification, recommendation or health indicator to the person. The LEDs may be configured to emit a first colour when the first-bio-parameter measurement is within acceptable limits and a second colour when a second bio-parameter measurement is needed. In some embodiments, only a single LED may be required. The LED may be configured to emit different light patterns in accordance with the bio-parameter measurements, such as solid or flashing. In some embodiments, the display may comprise a traffic light system with different colours representing different results.

In the embodiment shown in FIG. 4, first 42 and second 47 bio-parameter measurement devices are collocated and provided within a common housing 412. The housing 412 may be similar to that described in connection with FIG. 3 and will not be described further here.

The first bio-parameter measurement device 42 may comprise a first stage measurement of a particular bio-parameter measurement with the second bio-parameter measurement device 47 providing a second stage measurement in relation to the same bio-parameter measurement. As described above, the first stage measurement may be a non-contact and/or non-interactive bio-parameter measurement and the second stage measurement may be an interactive bio-parameter measurement.

In some embodiments, the first stage measurement may comprise a relatively coarse temperature measurement using an infrared detector comprising a thermal sensor such thermal imaging sensor for obtaining a temperature reading of a person within a field of view of the thermal sensor. Thus, the temperature reading may optionally be based on black-body radiation from the person. The temperature reading may be energy efficient and thus not result in a prolonged usage of a self-contained power source such as a battery. However, such a measurement may be generally inaccurate such that a person's temperature is not accurately provided. Thus, where the temperature is close enough to an acceptable limit of an upper (or possibly lower) temperature so as to be within expected margins of error for the type of temperature detector, the second bio-parameter measurement device may be utilised. The second bio-parameter measurement unit may, in some embodiments, comprise the same bio-parameter measurement device as the first bio-parameter measurement device 42, but require the person 5 to open their mouth such that a more accurate oral temperature may be obtained. In other embodiments, the second bio-parameter measurement device 47 may comprise the thermal camera and/or visible light camera described in connection with FIG. 2, for example, or some other form of detector.

It will be appreciated that in some embodiments, the first 42 and second 47 bio-parameter measurement device may share certain components. Hence, the first 42 and second 47 bio-parameter measurement devices may share the use of a visible camera for target location or feature recognition, but may use separate sensors to capture measurement data.

Although not shown, the system of FIG. 4 may comprise one or more additional first bio-parameter measurement devices 42 (e.g. sub-devices as described above), and/or one or more second bio-parameter measurement devices 47. The second bio-parameter measurement devices 47 may be wirelessly or hardwired connected as described in connection with FIG. 2.

Another optional feature shown in FIG. 4 is having the assessment module 43 and communications module 44 located remote from the first bio-parameter measurement device 42. Thus, the assessment module 43 and communication module 44 may be provided as part of a server 46.

The server 46 may be a conventional server and may be located remotely from the premises in which the bio-parameter measurement devices 42, 47 are located. The server 46 may be cloud based, for example, and connected to via a network 48. The server 46 may be a single device or a plurality of distributed devices. The first bio-parameter measurement device 42 may communicate with the server using any suitable network such as the Internet or other wide area network or a local area network, for example. Thus, the first bio-parameter measurement device may communicate via the wide area network using broadband mobile or via a wireless connection to an access point at the premises, using WiFi®, for example.

In a similar way, the communication module 44 may connect with the user device 410 via the network 48 using known methods. The communication with the mobile device 410 may be via an intermediate device. The intermediate device may, in some embodiments, reformat and/or manipulate the measurement or notification being sent to the mobile device. For example, the intermediate device may comprise a control hub or a server. The mobile device may use a first communication protocol and the intermediate device may use a different communication protocol.

It will be appreciated that, although the collocated first and second bio-parameter measurement devices 42, 47 are shown as being used in connection with the remote server in FIG. 4, this is not a limitation and other configurations of first 42 and second bio-parameters 47 (and other types) may be used to provide measurement data to a remote based server. Similarly, collocated bio-parameter measurement devices may be used with integrated assessment 43 and communication 44 modules. It will also be appreciated that in some embodiments, the communication module 44 which communicates with the user device may be remotely located with respect to the assessment module. Thus, the assessment module 43 may be located with the first and/or second bio-parameter measurement devices 42, 47 and may be configured to transmit the determination for a further bio-parameter measurement device to the communication module which sends the notification to the user device and awaits the validation.

FIG. 5 shows a user device in the form of a mobile device 51. The mobile device 51 may include one or more antenna 52 for external wireless communications and data transmissions; a user interface 53 comprising a display screen 54, speaker 55, and a microphone 56 with which a user can interact with the mobile device 51 to receive the notification or input a validation response; one or more applications 57 which may be configured to receive inputs or provide outputs which are associated with the notification or validation; a processor 58; a memory 59 which may store instructions which, when executed by the processor cause the performance of the method steps relevant to the mobile device 51; and a location device 510. The mobile device 51 may further comprise a camera for acquiring one or more visible light images. The images may be used to determine one or more bio-parameters using rPPG. As such, the user device 51 or a mobile device of the person may be used as the second bio-parameter measurement device 37.

The mobile device 51 may be any mobile computing device which is monitored regularly by a user. The mobile device 51 may be a mobile phone, a tablet computer or a laptop, for example. The features described in relation to the mobile device 51, may also be application to a user device which is not mobile, such as a desktop computer or workstation.

In the case of FIG. 5, the mobile device 51 is assumed to be a mobile phone in the form of a conventional smart phone which comprises conventional hardware and software to allow voice and text communications and data connections via various wireless communication channels such as mobile broadband, wireless local area networks, and short range wireless technologies such as near field communications and Bluetooth®, for example. The mobile device 51 is also configured to store and operate other applications, ‘apps’, as known in the art.

The display screen 54 may be a conventional touch screen which is configured to receive touch inputs from a user so as to allow the selection of a screen items such as menus or buttons. The screen items may be provided as part of the user interface 53 which may include options for a user to select, as part of an application or the notification, so as to provide a validation response. The validation response options may include one or more push buttons with predetermined responses attributed to them. For example, a first push button may be an “acknowledge” push button which transmits an acknowledgement that the notification has been received. A second button may be a “test carried out” push button which transmits a response that the second bio-parameter measurement has been carried out. A third button may be a “send” push button which configures the system to send the results of second bio-parameter measurement, i.e. the second measurement data, to the communication module by way of validation. The second measurement data may be held by the mobile device or may be held elsewhere, e.g. the second bio-measurement device.

The notification may be a push notification and may be provided as an on-screen dialogue box or a dropdown menu. Alternatively, the notification may cause the application 57 to launch upon receipt of the notification. The application 57 may provide a user interface display with the push buttons and other functionality in relation to the system.

In addition to or as an alternative, the notification may provide an audible output via the speaker 55. The audible output may be an alert noise such as a beep to flag the receipt of the notification to a user 6, or may provide a spoken word version of the notification. The audible output may additionally provide spoken word validation response options for a user to select from. The microphone may receive spoken user validation responses which are processed by the user device as known in the art.

The memory 59 may comprise a non-transitory computer readable storage medium comprising computer readable instructions that, when read by a processor of a mobile phone, configure the mobile phone to carry out the methods described herein. The computer readable instructions may comprise executable code relating to the application, for example. Thus, the memory may comprise one or more computer programs which, when executed by the mobile device 51, cause the performance of the methods described herein in relation to the notifications and/or validation responses. The memory may comprise: volatile memory, for example, one or more dynamic random access (DRAM) modules and/or static random access memory (SRAM) modules; and/or non-volatile memory, for example, one or more read only memory (ROM) modules, which for example may comprise a Flash memory and/or other electrically erasable programmable read-only memory (EEPROM) device. In other embodiments the data store may be separate from the mobile device 210. For example, it may be located in a memory component of a remote server or a portable memory device such as a memory card, e.g. SD card or microSD®. Alternatively, the data store may be distributed amongst a plurality of devices, for example, partly on the mobile device 210 and partly remotely).

The processor 58 may be a conventional mobile device processor which may include one or more microprocessors executed in hardware or software, for example. The processor may include at least one microprocessor and may comprise a single core processor, may comprise multiple processor cores (such as a dual core processor or a quad core processor), or may comprise a plurality of processors (at least one of which may comprise multiple processor cores). In various examples, the processor may comprise: control circuitry; and/or processor circuitry; and/or at least one application specific integrated circuit (ASIC); and/or at least one field programmable gate array (FPGA); and/or single or multi-processor architectures; and/or sequential/parallel architectures; and/or at least one programmable logic controllers (PLCs); and/or at least one microprocessor; and/or at least one microcontroller; and/or a central processing unit (CPU); and/or a graphics processing unit (GPU), and/or transceiver(s) to perform the methods.

The bio-parameter measurement devices, assessment modules and communication modules described herein may respectively comprise one or more processors and one or more memories. The processors, or at least a part thereof, may be implemented hardware or software and may be implemented as a controller, rather than a processor per se. The processors may comprise, for example: control circuitry; and/or processor circuitry; and/or at least one application specific integrated circuit (ASIC); and/or at least one field programmable gate array (FPGA); and/or single or multi-processor architectures; and/or sequential/parallel architectures; and/or at least one programmable logic controllers (PLCs); and/or at least one microprocessor; and/or at least one microcontroller; and/or a central processing unit (CPU); and/or a graphics processing unit (GPU), and/or transceiver(s) to perform the methods described herein.

The memory devices may store code/instructions that, when read by the processor, causes performance of any of the methods described herein, and/or as illustrated in in the drawings. For example, the memory may comprise: volatile memory, for example, one or more dynamic random access (DRAM) modules and/or static random access memory (SRAM) modules; and/or non-volatile memory, for example, one or more read only memory (ROM) modules, which for example may comprise a Flash memory and/or other electrically erasable programmable read-only memory (EEPROM) device. The code may for example be software, firmware, or hardware description language (HDL) or may be any combination of these or any other form of code for one or more processing devices that is known by a person skilled in the art.

The terms “comprising” and “including”, provided that is the intended scope of these terms. An example of a suitable statement is: “As used herein, except where the context requires otherwise, the terms “comprises”, “includes”, “has”, and grammatical variants of these terms, are not intended to be exhaustive. They are intended to allow for the possibility of further additives, components, integers or steps.”

It will be understood that the invention is not limited to the examples and embodiments above-described and various modifications and improvements can be made without departing from the concepts described herein. Except where mutually exclusive, any of the features may be employed separately or in combination with any other features and the disclosure extends to and includes all combinations and sub-combinations of one or more features described herein.

In addition, any priority document(s) of this application is/are hereby incorporated herein by reference in its/their entirety.

1. A bio-parameter measuring system comprising:
a first bio-parameter measurement device for performing a first bio-parameter measurement of a person without physical contact with the person;
an assessment module configured to:
receive first measurement data from the first bio-parameter measurement device; and,
determine whether a second bio-parameter measurement is required on the basis of the first measurement data; and
a communication module configured to:
upon a positive determination that a second bio-parameter measurement is required, notify a user that a second bio-parameter measurement is required; and,
receive a validation that the user has undertaken the second bio-parameter measurement,
wherein the first bio-parameter measurement device comprises a radar or sonar.

2. The system of claim 1, further comprising a second bio-parameter measurement device for providing the second bio-parameter measurement.

3. The system of claim 2, wherein the validation comprises one or more from the group comprising:
a confirmation received from the user;
a confirmation received from the second bio-parameter measurement device;
and,
second measurement data relating to the second bio-parameter measurement received from the second bio-parameter measurement device.

4. (canceled)

5. The system of claim 1, further comprising a user device for receiving a notification that a further bio-parameter measurement is needed.

6. (canceled)

7. The system of claim 5, wherein the user device comprises a user interface having one or more input devices for inputting a validation response for transmission to the communication module as the validation, wherein the one or more input device comprises either or both of: a push button or a microphone for receiving a voice command.

8. The system of claim 1, wherein one or more or all of: the first bio-parameter measurement device; the assessment module; and the communication module are within a single unit.

9.-13. (canceled)

14. The system of claim 1, further comprising a detector for detecting a presence or motion of a person in the vicinity of the first bio-parameter measurement device, wherein first bio-parameter measurement device is configured to carry out the first bio-parameter measurement following an output signal from the detector.

15. (canceled)

16. The system of claim 1, wherein the first bio-parameter measurement device further comprises one or more or all of:
a camera for obtaining one or more images of the person in the visible spectrum;
an infrared detector.

17. The system of claim 1, wherein the first bio-parameter comprises respiratory rate.

18. The system of claim 2, wherein the second bio-parameter measurement device comprises a pulse oximeter.

19.-21. (canceled)

22. The system of claim 1, wherein the second bio-parameter measurement requires an action from the person.

23. The system of claim 1, wherein notifying the user that a second bio-parameter measurement is required comprises providing a prompt for the user.

24. A method of measuring a bio-parameter of a person comprising:
obtaining, using a first bio-parameter measurement device, first bio-parameter measurement data of the person without physical contact with the person;
receiving, at an assessment module, first measurement data from the first bio-parameter measurement device;
determining whether a second bio-parameter measurement is required on the basis of the first measurement data;
upon a positive determination that a second bio-parameter measurement is required, notifying, using a communication module, a user that a second bio-parameter measurement is required; and,
receiving, at the communication module, a validation that the user has undertaken the second bio-parameter measurement,
wherein the first bio-parameter measurement device comprises a radar or sonar.

25. The system of claim 1, comprising:
a user interface and a processor configured to receive a notification that a second bio-parameter measurement is required from a person, output one or more validation response options using the user interface for a user of the computing device to select and, upon receiving a selection of the one or more validation response options from the user, transmit the selected validation response to the bio-parameter measurement system.

26. The computing device of claim 25, wherein the one or more validation response options include one or more or all of: an acknowledgement that the notification has been received; an option to confirm that the required bio-parameter measurement has been carried out; an option to transmit the bio-parameter measurement; and an option to confirm that the required bio-parameter measurement has been carried out.

27. (canceled)

28. The method of claim 24, wherein the first bio-parameter measurement device further comprises one or more or all of:
a camera for obtaining one or more images of the person in the visible spectrum;
an infrared detector; and
a thermal imaging device.

29. The method of claim 24, wherein the first bio-parameter comprises respiratory rate.

30. The method of claim 24, further comprising obtaining the second bio-parameter measurement from a second bio-parameter measurement device and wherein the second bio-parameter measurement device comprises a pulse oximeter.

31. The method of claim 24, wherein upon receiving, at a computing device, a notification that a second bio-parameter measurement is required from a person, outputting one or more validation response options using a user interface for a user to select and, upon receiving a selection of the one or more validation response options from the user, transmitting the selected validation response to the communication module.

32. The method of claim 24, wherein the one or more validation response options include one or more or all of: an acknowledgement that the notification has been received; an option to confirm that the required bio-parameter measurement has been carried out; an option to transmit the bio-parameter measurement; an option to confirm that the required bio-parameter measurement has been carried out.
